(19) United States
(12) Patent Application Publication
(10) Pub. No.: US 2018/0126116 A1
(43) Pub. Date: May 10, 2018

(54) Integrated Stroboscopic Eyewear For Sensory Training

(71) Applicant: Benjamin L. White, Rahway, NJ (US)

(72) Inventor: Benjamin L. White, Rahway, NJ (US)

(73) Assignee: BLW IP, LLC, Rahway, NJ (US)

(21) Appl. No.: 15/804,193

(22) Filed: Nov. 6, 2017

Related U.S. Application Data
(60) Provisional application No. 62/418,649, filed on Nov. 7, 2016.

(51) Int. Cl. A61M 21/00; G02F 1/1334; G09B 5/00; G09B 19/00

(52) U.S. Cl. CPC A61M 21/00; G09B 19/00; G09B 5/00; G02F 1/1334

(57) ABSTRACT

A wireless, programmable and customizable, integrated stroboscopic, eyewear device used as a training tool to enhance the user's sensory processing systems and overall neuro capability (including the underlying pyscho-motor functions). This device functions to elevate and improve the user's perceptual and cognitive processing skills and further measures and collects sensory data from the user during training including heart rate and other Bio statistics. It overall provides for a sensory training tool that builds and strengthen the user's confidence and mental state (ability to spontaneously adjust and respond to unanticipated events). The eyewear device is communicatively coupled to a computing or communication device to transmit data to the receivers. The eyewear device is either equipped with a Bluetooth, infra-red, or Wi-Fi communications module to enhance the connectivity of the device and to oversee the performance of the user using a mobile application resident on a remote mobile device such as a tablet computer or a smartphone.

### CROSS REFERENCE TO RELATED APPLICATION

This application claims benefit of U.S. Provisional Application No. 62/418,649 filed on 7 Nov. 2016 which is incorporated by reference.

### TECHNICAL FIELD OF THE INVENTION

The present disclosure relates to applications of the stroboscopic effect. More particularly, the present disclosure relates to an integrated stroboscopic eyewear device. This eyewear device is a sensory training tool to be used to train and sharpen the wearer's sensory abilities including communication between the eyes, mind and body for purposes of improving athleticism, skills enhancement, physical rehabilitation, decision-making and executive functioning enhancement.

### BACKGROUND OF THE INVENTION

Eyes are similar to a pair of synchronized cameras that constantly receive visual information. All information entering the eyes is converted into signals that are sent to the brain for processing. Because of this, it is apparent that perception of images occurs in the brain, rather than in the eyes. The brain then feeds instructions back to the eyes and also to other parts of the body in response to the information it receives. While the entire process occurs at unimaginable speed, there is still a measurable lapse of time between receipt of visual information at the eyes and the receipt of instructions back from the brain at the eyes and the other body parts.

Vision training, or vision therapy, has been known, at least, since the 19^{th} century. In the late 19^{th} century, French ophthalmologist Louis Emile Javal pioneered the use of ocular exercises for treatment of strabismus.

In the early 20^{th} century, an eye-care physician, William Bates, developed the Bates Method of Eyesight Improvement. These early methods of eyesight improvement focused on the eye itself, and the musculature of the eye to correct muscle dysfunction and refractive defects.

While early practitioners of vision improvement understood the connection between the central nervous system and the eyes, their approaches to vision improvement were directed exclusively to the eyes, completely ignoring the role of the central nervous system in vision.

Unbeknownst to the early practitioners, vision training causes enhancements beyond just vision improvement. Vision training, as later discovered, improves the efficiency with which the central nervous system processes sensory input. This was a big oversight. The benefits and effect from vision training on the user/trainee can lead to improvements in learning disabilities, executive and cognitive functioning skills, and athletic performance, and more, all achievable through vision and other types of sensory training.

These benefits and effect referenced above include, without limitation, enhanced sensory integration (coordination), concentration (focus) and psycho-motor reaction time, which are integral to many professions and occupations. For example, athletes, first responders, warfighters, law enforcement, and healthcare workers must have quick mental and physical reflexes to meet the demands of their chosen profession. To address the need for increased sensory integration, mind body control, and more proficient reflexes, a large number of products have been designed and marketed to the aforementioned professionals.

Various types of vision training eyewear have been proposed to produce improvements in human performance, such as improving eye-hand coordination. Typically, such devices have lenses that limit the field of vision in some way, through the provision of slots and/or apertures. The slots and/or apertures are provided, for example, to help the wearer develop his/her ability to maintain focus on a certain point.

Other vision training devices use lenses of varying color to produce selected training effects. For training, one example uses glasses having green lenses to make the red stitching on a baseball stand out. There, the exercise trains the wearer to detect the ball's spin.

The existing products generally function by blocking a user's sight for preset time increments, limiting the amount of time in which an object is visible and therefore decreasing the amount of time a user has to react to the object. The concept is that users will gradually improve their reflexes to adapt to the reduced time in which an object is visible.

Newer forms of sports training eyewear use various technologies to adjustably modify the optical transmittance of an entire lens or of predetermined regions of the lens. Some examples of this type of eyewear use liquid crystal technology to vary the optical transmittance of the lens. Other examples use electrically switchable optical materials to produce flickering dynamic images. Various forms of training effect are induced in this way. Conventional liquid crystal technology requires that the liquid crystals be fabricated on a glass substrate. The use of glass substrate, however, imposes limitations on the shape of the liquid crystal lens, requiring, essentially, that the lenses be flat. The current eyewear products have only limited programmability, in part, owing to the necessity to do reload new firmware in order to do program updates and also due to limited onboard storage. Also, current products are limited either to local control and/or remote control via a wired connection.

Taking into account the advantages of vision training and the limitations that the prior arts vision training eyewear have, there is a standing need of an integrated stroboscopic eyewear that can be used to train and sharpen the wearer's sensory abilities better performance of athletes.

### SUMMARY OF THE INVENTION

Present invention discloses a Programmable, integrated stroboscopic eyewear that could train the visual system in all aspects of visual capability. The present eyewear device can be used to improve the perceptual and cognitive skills, and the reflexes, further improves coordination and balance required for peak performance by athletes, warfighters, pilots and astronauts, vehicle and bike operators, first responders, surgeons, industrial engineers and calibration inspectors, stock traders, and law enforcement. The device can be used as an enhancement tool in physical rehabilitation, mitigating learning disabilities, and improving executive functioning skills and executive decision-making. As an added integrated feature, the device contains a sensor for capturing the wearer's heart rate and other vitals during use.

In one embodiment of the invention, under control via an integrated UI, liquid crystal display drivers allow the device's conforming lenses to switch between opaque and transparent states. Through the use of this stroboscopic technology, the eyeglasses, as programmed, intermittently limit the amount of visual information received through the lenses. The brain is then compelled to compute the last received information and to create images during the blackout periods from the last received visual information, thereby compensating for the lack of visual information being received during the blackout. Users may experience cognitive/perceptual enhancements such as improved object vision and threat identification; visual vigilance; situational awareness; recovery from surprise and loss of focus; adaptability to changing environmental demands; reduction in information overload and confusion; decreased visual blindness and unintentional blindness; decision-making, overall cognition and motor memory, and mental toughness.

How the foregoing objectives are achieved will be clear from the following description. In this context, it is clarified that the description provided is non-limiting and is only by way of explanation.

### BRIEF DESCRIPTION OF THE DRAWINGS

The nature and scope of the present invention will be better understood from the accompanying drawings, which are by way of illustration of a preferred embodiment and not by way of any sort of limitation. In the accompanying drawings:—

FIG. 1 provides a functional block diagram of an integrated stroboscopic eyewear device;

FIG. 2 represents a perspective view of the integrated stroboscopic eyewear device having control buttons and LED display;

FIG. 3 illustrates the attachment of strap and corresponding connection element with the integrated stroboscopic eyewear device;

FIG. 4 represents a communication device used to remotely control the integrated stroboscopic eyewear device;

FIG. 5 illustrates a computational device that coordinates functional aspect of remotely controlling the integrated stroboscopic eyewear device;

FIG. 6 shows the diagram of the eyewear device communicatively being coupled to a communications device as shown in FIG. 4.

### DETAILED DESCRIPTION

In the following paragraphs, a brief and non-limiting description of the preferred embodiment is disclosed.

All through the specification, the technical terms and abbreviations are to be interpreted in the broadest sense of the respective terms, and include all similar items in the field known by other terms, as may be clear to persons skilled in art. Restriction or limitation if any referred to in the specification, is solely by way of example and understanding the present invention. Ranges may be expressed herein as from ‘about’ or ‘approximately’ another particular value. Also, it will be understood that unless otherwise indicated, dimensions and material characteristics stated herein are by way of example rather than limitation, and are for better understanding of sample embodiment of suitable utility and variations outside of the stated values may also be within the scope of the invention depending upon the particular application. Exemplary embodiments of the disclosure as described herein generally include an integrated stroboscopic eyewear that could train the visual system in all aspects of visual capability. Accordingly, while embodiments of the disclosure are susceptible to various modifications and alternative forms, specific embodiments thereof are shown by way of example in the drawings and will herein be described in detail. It should be understood, however, that there is no intent to limit embodiments of the disclosure to the particular exemplary embodiments disclosed, but on the contrary, embodiments of the disclosure cover all modifications, equivalents, and alternatives falling within the spirit and scope of the disclosure.

The present invention seeks to address the drawbacks associated with the existing programmable Vision training eyewear that either has limited onboard storage or are limited to local control or wired connection control. To achieve this purpose, Present Invention discloses an unlimited programmable integrated device that is controlled via an integrated UI and has a liquid crystal display drivers that allow conforming lenses to switch between opaque and transparent states at the user's desired frequency.

The presently-described device advances the application of stroboscopic technology in the form of a fully integrated eyewear device equipped with/comprises:

(i) stroboscopic lenses of variable colors with opaqueness adjustment and control system (a variable opaqueness control);
(ii) device embedded HRV (heart rate variability) sensors;
(iii) integrated micro speakers (bone conduction) for feedback signaling;
(iv) presets and expandable programming capability for user customization including downloadable training apps and programs; and
(v) user-trainer wireless communication and device control operated via WiFi, Bluetooth or infrared.

In one of the embodiments of the present invention, the eyewear device may use a special display technology having polymer-dispersed liquid crystals (PDLC), to create a stroboscopic effect. The PDLC lens may be fabricated from a material that includes a solid polymer matrix having liquid crystal droplets disposed with the cells of the matrix. In another embodiment, the polymer matrix may be deposited on a conforming polymer substrate, such as polyethylene terephthalate (PET). The droplets as disclosed herein may be in the micron-size scale. So that the Micron-size scale gives the lens its unique capabilities.

Use of an electric field to change the orientation of the liquid crystal molecules within the matrix makes it possible to alter the optical transmittance of the lens, or of selected portions of the lens, in predetermined ways.

The lenses may be fabricated from scattering PDLCs, wherein, the ‘off’ state, the PDLCs are randomly oriented to each other. In this random-oriented state, the PDLCs are in a highly optical-scattering state, resulting in a lens fabricated therefrom, being in an opaque state. And conversely, in the ‘on’ state, when the PDLCs are subjected to an electric field, the PDLC's may reorient to each other electro-optically so that scattering is reduced, with a transparent state resulting. Thus, by using scattering PDLCs having micron-sized droplets such as this, in the embodiment, lenses produced from such material have the capability of cycling rapidly between opaque and transparent states.

Within the foregoing embodiment, chemical dyes, for example red, green or blue light, may be added to the PDLC mixture, so that it may preferentially scatter.

Furthermore, in another embodiment, the droplet surface area may be optimized to produce an extremely rapid switching time. The eyewear device may be provided with multicolored/dye-based stroboscopic lenses for signature eyewear lines. Thus, the eyewear device, when turned ON, supplies power to the lens in a cyclic fashion, causing the lens to rapidly or steadily cycle between transparent and opaque, as energy is alternately supplied to and withdrawn from the PDLCs, resulting in a shutting ON and OFF effect to the eye. On the other hand, when the device control is in OFF position, power is withdrawn from the PDLCs, allowing them to return to their random state resulting the lenses to return to their default, opaque condition.

In embodiments, the frequency of this open/close sequence, may be varied using one or more integrated controls on the eyewear device by varying the length of the duty cycle, with an occlusion effect resulting. Through the use of this stroboscopic effect, the eyewear device intermittently limits the amount of visual information received through the lenses, resulting in repeated occlusion periods. The user's brain is thus compelled to create images during the occluded periods from the last received visual information, thereby compensating for the lack of received visual information during the occluded state. One salutary effect of the temporal occlusion effect is that the user's mind and body synchronize from the enhanced visionary processing thus, making the user's concentration, visual memory and motor reflexes sharper and more efficient.

In a preferred embodiment, the eyewear device may be equipped with a Bluetooth, infra-red, or Wi-Fi (described in IEEE 802.11, the entirety of which is incorporated herein by this reference) communications module to enhance the connectivity of the device and to oversee the performance of the user using a mobile application resident on a remote mobile device such as a tablet computer or a smartphone. In embodiments, the Wi-Fi communication module may be an integrated Wi-Fi transceiver.

The mobile application (app) may also be used to control and vary occlusion speed of the stroboscopic PDL or LCD lenses. The device may be powered via a rechargeable lithium-battery pack. The battery pack maybe recharged using a pin-port or micro-USB connection.

Further, the eyewear device comprises a frame and a pair of lenses, and heart rate sensors, bone conduction transducers and Haptic sensors. Wherein the frame comprises a first lens support, a second lens support, a bridge, a first temple, and a second temple. The bridge is coplanar with the first lens support and the second lens support, which are connected to each other by said bridge. The first temple is connected perpendicular to the first lens support, while the second temple is connected perpendicular to the second lens support. The first temple and the second temple support secure the eyewear device to a user by resting on the user's ears. Likewise, the bridge helps hold the eyewear device in place by resting upon the user's nose. The pair of lenses comprises a first lens and a second lens, which are respectively supported by the first lens support and the second lens support. More specifically, the first lens support is connected around the first lens just as the second lens support is connected around the second lens.

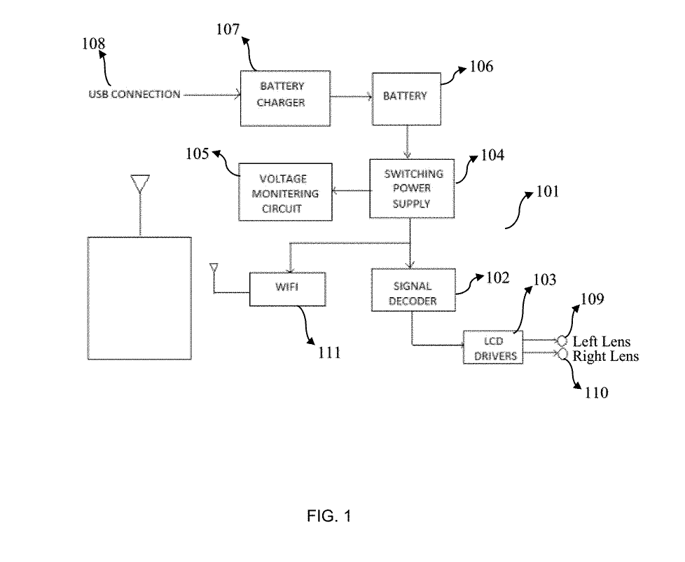

Referring particularly to FIG. 1, the functional process 101 of the eyewear device 201 is shown. The eyewear device as shown may include at least one of a plurality of electronic modules such as:
A power control;
One or more displays in the form of stroboscopic PDLC or LCD lenses;
Communications;
A processor;
A mobile app;
Heart Rate Sensors
Bone Conduction Transducers
Haptic Sensors

In order to use the variable lenses, the eyewear device further includes an electronics package, as detailed in FIG. 1. The electronics package comprises a wireless communication module 101, a signal decoder 102 and liquid crystal display (LCD) and PDL drivers 103. Powering the electronics package is a power system, which itself comprises a power source, a switching power supply 104, and a voltage monitoring circuit 105. In an embodiment, the wireless communication module 101 may be a RF receiver, while the power source is a battery 106. In addition, the power system further includes a battery charger 107, provided for recharging the battery. The battery maybe recharged using a pin-port or micro-USB connection 108. In an embodiment, the power system may be electrically connected to the electronics package, as well as the pair of lenses, supplying the necessary energy to operate the components of the eyewear device. The LCD/PDL drivers are electronically connected to the first lens 109 and the second lens 110.

In an embodiment, the power control is operative to supply required power to the different modules via a rechargeable battery pack installed in the device. In an embodiment, this module may have, as shown in FIG. 1, at least one of:
Power management circuitry to supply power to other components.
Battery pack to provide power; and
One or more charging & protection units to monitor and protect the device from high currents and voltages. In an embodiment, a charge controller and protection unit is operative to protect the battery pack and charge it via a charging port.

In an embodiment, the processing module is operative to control all other modules and monitor execution of their respective tasks. In an embodiment, the processing module may include at least one of:
A processor;
A memory; and
A user interface to by which a user may interact with the device.

In an embodiment, the power management circuitry may include a power management integrated control chip to distribute the required power. In an embodiment, power management circuitry provides an optimized path to power the devices;

In an embodiment, the displays, or lenses, may be controlled by the processor and powered by the power control module. This module is operated directly by the processor. In an embodiment, the displays or lenses may constitute one or more polymer-dispersed liquid crystal (PDLC) displays.

In an embodiment, the communications module is controlled by the processing unit and receives inputs from the communication device 401 or computing device 501. The communications module may have Wi-Fi connectivity 111 and leverage an existing Wi-Fi network. This module sends all its received info to the processor to process and perform actions. In an embodiment, the communications module may include a Wi-Fi—wireless connectivity microchip. Wi-Fi connectivity provides an alternate and ‘connected-devices’ option to control the device over the air, leveraging existing wireless networks.

The wireless remote control capability also enables an embodiment wherein multiple eyewear devices may be networked with a single wireless computing device, thereby allowing synchronous control of each eyewear device via an app running on the computing device.

In an embodiment, the eyewear device is equipped with onboard RAM (random access memory), which gives the device the ability to easily download and store control programs, eliminating the necessity of reloading firmware. Additionally, the ability to download, store and run programs allows a great range of programming options, limited only by the imagination of the programmer. The eyewear device may be programmable with customizable firmware allowing for a multiplicity of selectable pre-sets. In an embodiment, the eyewear device may have by provided with embedded expandable memory and data storage capacity.

In an embodiment, the mobile app is operative to control shutter speeds for the visual device and monitor the performance of the user over time.

In an embodiment, the eyewear device is equipped with a dual-control system. In a first aspect, the dual control system allows remote, programmatic control of the eyewear device from a communication 401 or computational device 501 to which the eyewear device is communicatively coupled via a wireless data connection.

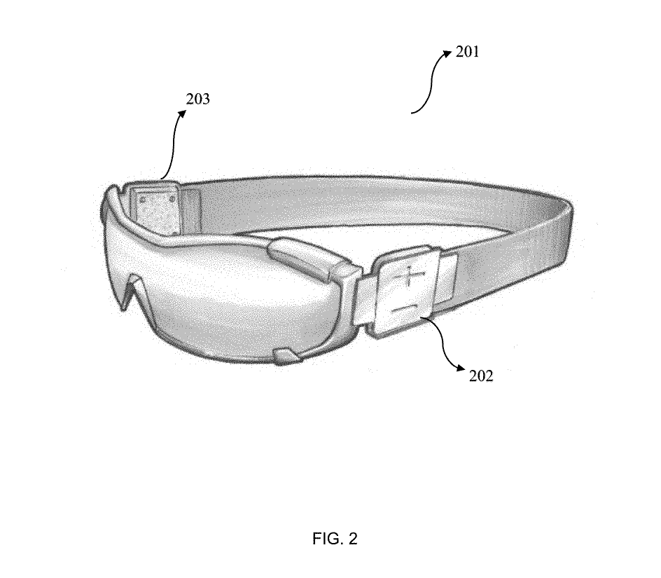

FIG. 2 shows a perspective view of the eyewear 201 having one or more on-board control buttons 202 and a display 203 are provided to enable control and operation of the eyewear device by the user. The on-board control system may include one or more of:
One or more touch-sensitive buttons 202 that allow the user to vary the strobe levels by selecting from as many as 50 or more different programs;
At least one LCD screen 203, 301 that display program information;
At least one LED light indicator 302 that may be operative to indicate an on/off state and/or a low battery.

The control display is separate from the PLCD lenses. In an embodiment, the control interface may be integrated within one or both of the lenses.

In an embodiment, the different components mentioned herein above may be connected physically either via a printed circuit boards or by wires. In an embodiment, the various modules interact via paths laid on the printed circuit boards, under control of the processor. The physical arrangement of the different modules is done so as to maintain the equilibrium of the whole product and to utilize the available space as efficiently as possible.

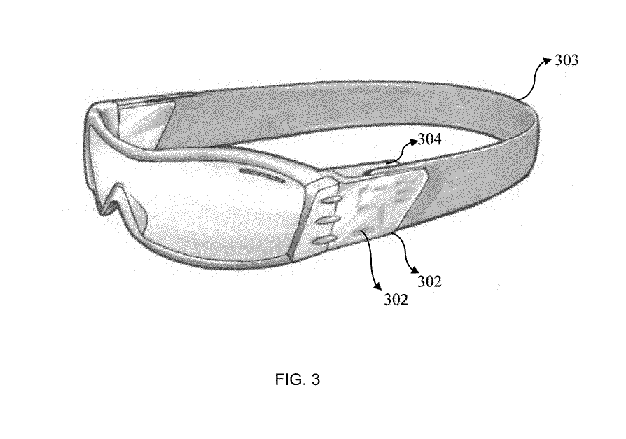

FIG. 3 illustrates the accessorial components of the eyewear device that includes a strap 303 and a corresponding connection element such as buckle or a latch or a fastener 304. In embodiments, the strap 303 may be operative as head strap to secure the eyewear device in place during use. The strap 303 and connection element 304 may also be operative as an armband for securely carrying the eyewear device on the user's body when not in use.

Materials from which the eyewear device is fabricated may without limitation include, high-density polypropylene or polyethylene. As described above, the lenses may be fabricated from a polymer having dispersed therein a plurality of micron-scale liquid crystals. In another embodiment, the polymer-dispersed liquid crystals may be manufactured on substrate formed from a conforming polymer, such as polyethylene terephthalate (PET), which permits the lenses to be formed into a variety of shapes, for example, curved lenses, or even goggles. The electrical components of the eyewear device may be fabricated primarily from silicon and other polymers as well.

These stated materials are durable sufficiently to stand up to the rigors of indoor and outdoor physical and athletic training and other rough-and-tumble training scenarios or drills. Having materials that are shatterproof and/or able to provide a hermetic seal for the inner components (silicon pieces) enables protection of the inner processing and power-supply units, as well as the lenses. Further, the electrical components and lenses of the eyewear device are so thoroughly shielded that the device is water-resistant to provide a water-Resistant stroboscopic eyewear device.

While an embodiment of the eyewear device utilizes a standard eyeglass design, other embodiments may take the form of a pair of goggles and a visor, with adjustments being made as necessary. For example, the visor, instead of the two-lens configuration employed in the glasses and goggles, uses a single lens. In a further embodiment of the visor, two adjacent lenses are joined with a bezel. Other eyewear designs can be used in further embodiments as long as they are capable of utilizing the voltage controlled liquid crystal lens, the Wi-Fi receiver and the relevant supporting components (e.g. the power system) described herein.

In an embodiment, the eyewear device is provided with high-opacity LCD or PDLC lenses, which allows the eyewear device to block out a greater percentage of visual information when the lenses strobe, in comparison with conventional eyewear devices. The device also contains a control allowing the user to adjust the opacity of the LCD or PDLC lenses ranging from high-opacity for some exercises (outdoor activities or increasing difficulty), to less for others (indoor activities).

Embodiments of the eyewear device may be compatible with any of communication devices 401 such as iPhone, Android, Windows, Amazon, and most other wireless devices.

Embodiments of the eyewear device are provided with lenses having a variety of colors appropriate for the training environment or per the user's desired personal taste and selection.

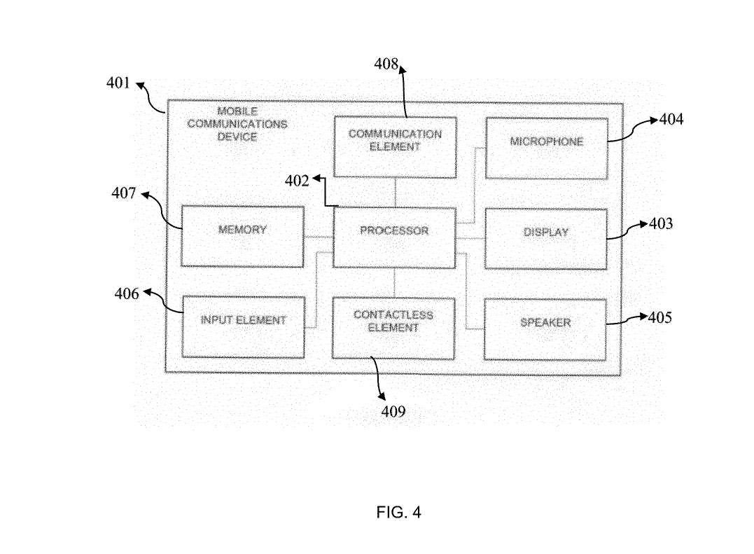

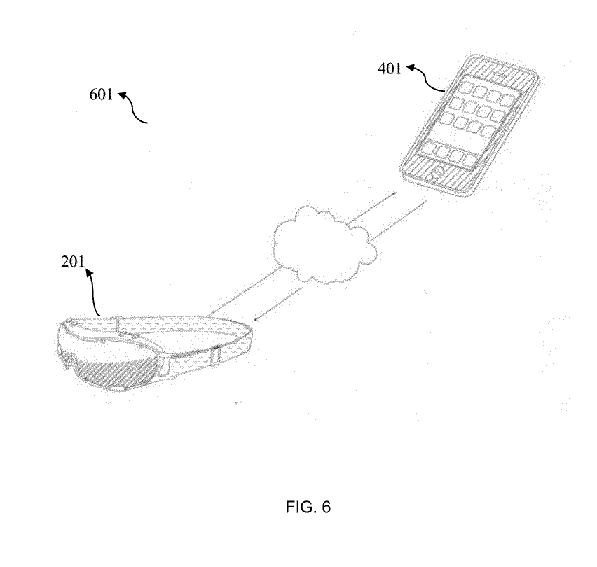

FIG. 4 shows a block diagram of the mobile communication device 401 that may be used with embodiments of the eyewear device in a system for sensory training as shown in FIG. 6. The communication device 401 may be a cell phone, a feature phone, a smart phone, a satellite phone, or a computing device having wireless communication capability.

The communications device 401 may include a processor 402 (e.g., a microprocessor) for processing the functions of the communications device and a display 403 to allow a user to see the phone numbers and other information and messages. Further, Microphone 404 and speaker system 405 are placed to enable communication with the device and other subsystems. The communications device may further include an input element 406 to allow a user to input information into the device (e.g., input buttons, touch screen, etc.).

The processor of the communications device may connect to a memory 407. The memory 407 may be in the form of a computer-readable medium that stores data and, optionally, computer-executable instructions.

The communications device may also include a communication element 408 for connection to communication channels (e.g., a cellular telephone network, data transmission network, Wi-Fi network, satellite-phone network, Internet network, Satellite Internet Network, etc.). The communication element may include an associated wireless transfer element, such as an antenna.

The communication element 408 may include a subscriber identity module (SIM) in the form of an integrated circuit that stores an international mobile subscriber identity and the related key used to identify and authenticate a subscriber using the communications device. One or more subscriber identity modules may be removable from the communications device or embedded in the communication device.

The communications device 401 may further include a contactless element 409, which is typically implemented in the form of a semiconductor chip (or other data storage element) with an associated wireless transfer element, such as an antenna. The contactless element 409 may be associated with (e.g., embedded within) the communications device and data or control instructions transmitted via a cellular network may be applied to the contactless element 409 by means of a contactless element interface (not shown). The contactless element 409 interface may function to permit the exchange of data and/or control instructions between mobile device circuitry (and hence the cellular network) and the contactless element.

The contactless element 409 may be capable of transferring and receiving data using a near field communication (NFC) capability (or near field communications medium) typically in accordance with a standardized protocol or data transfer mechanism (e.g., ISO 14443/NFC, the entirety of which is incorporated herein in its entirety by this reference thereto). Near field communications capability is a short-range communications capability, such as radio-frequency identification (RFID), Bluetooth, infra-red, or other data transfer capability that can be used to exchange data between the communications device and an interrogation device. Thus, the communications device may be capable of communicating and transferring data and/or control instructions via both a cellular network and near field communications capability.

The data stored in the memory 407 may include: operation data relating to the operation of the communications device 401, personal data (e.g., name, date of birth, identification number, etc.), vitals, etc. A user may transmit this data from the communications device to selected receivers.

In another embodiment, the communications device 401 may be, amongst other things, a notification device that can receive alert messages and access reports, as well as a portable consumer device.

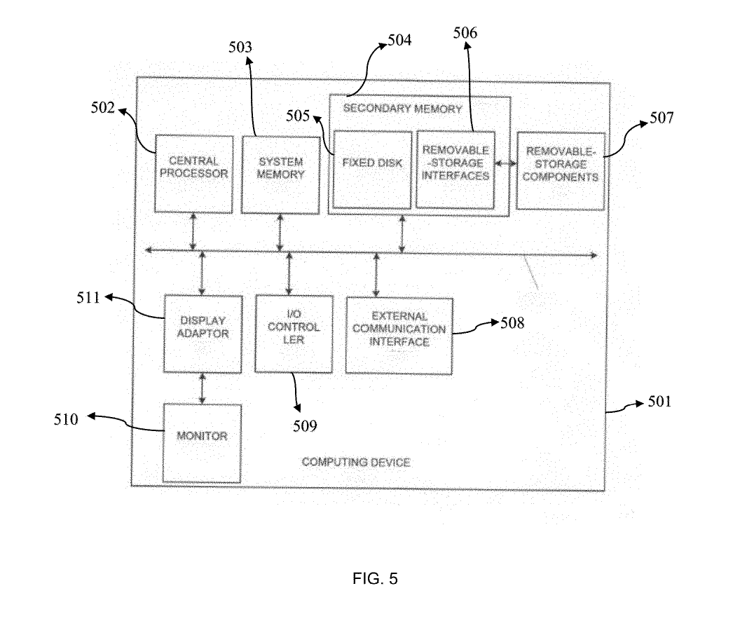

FIG. 5 illustrates an example of a computing device 501 in which various aspects of the disclosure may be implemented. The computing device 501 may be suitable for storing and executing computer program code. The various participants and elements in the previously described system diagrams may use any suitable number of subsystems or components of the computing device to facilitate the functions described herein.

The computing device may include subsystems or components interconnected via a communication infrastructure (for example, a communications bus, a cross-over bar device, or a network). The computing device may include at least one central processor 502 and at least one memory component in the form of computer-readable media.

The memory components may include system memory 503, which may include read only memory (ROM) and random access memory (RAM). A basic input/output system (BIOS) may be stored in ROM. System software may be stored in the system memory including operating system software.

The memory components may also include secondary memory 504. The secondary memory 504 may include a fixed disk 505, such as a hard disk drive, and, optionally, one or more removable-storage interfaces 506 for removable-storage components 507.

The removable-storage interfaces 506 may be in the form of removable-storage drives (for example, magnetic tape drives, optical disk drives, floppy disk drives, SDHC card, etc.) for corresponding removable storage-components 507 (for example, a magnetic tape, an optical disk, a floppy disk, etc.), which may be written to and read by the removable-storage drive.

The removable-storage interfaces 506 may also be in the form of ports or sockets for interfacing with other forms of removable-storage components, such as a flash memory drive, external hard drive, or removable memory chip, etc.

The computing device 501 may include an external communications interface 508 for operation of the computing device 501 in a networked environment enabling transfer of data between multiple computing devices. Data transferred via the external communications interface 508 may be in the form of signals, which may be electronic, electromagnetic, optical, radio, or other types of signal.

The external communications interface 508 may enable communication of data between the computing device 501 and other computing devices including servers and external storage facilities. Web services may be accessible by the computing device via the communications interface.

The external communications interface 508 may also enable other forms of communication to and from the computing device including, voice communication, near field communication, Bluetooth, etc.

The computer-readable media in the form of the various memory components may provide storage of computer-executable instructions, data structures, program modules, and other data. A computer program product may be provided by a computer-readable medium having stored computer-readable program code executable by the central processor.

A computer program product may be provided by a non-transient computer-readable medium, or may be provided via a signal or other transient means via the communications interface.

Interconnection via the communication infrastructure allows a central processor 502 to communicate with each subsystem or component and to control the execution of instructions from the memory components, as well as the exchange of information between subsystems or components.

Peripherals (such as printers, scanners, cameras, or the like) and input/output (I/O) devices (such as a mouse, touchpad, keyboard, microphone, joystick, or the like) may couple to the computing device 501 either directly or via an I/O controller 509. These components may be connected to the computing device by any number of means known in the art, such as a serial port.

One or more monitors 510 may be coupled via a display or video adapter 511 to the computing device.

FIG. 6 shows an overview 601 of the system for sensory training that includes at least one eyewear device as shown in FIG. 2 and FIG. 3 communicatively coupled with a communication device as shown and described above in relation to FIG. 4. While the exemplary embodiment of FIG. 6 shows a single eyewear device 201 communicatively coupled with the communication device 401. In actual fact, a plurality of eyewear devices may be communicatively coupled with the communication device 401 in order to perform sensory training sessions with a plurality of trainees in a single session, as described herein above. While FIG. 6 shows an embodiment of the system that includes coupling with the communication device 401, embodiments may, instead of the communication device, may couple the eyewear device 201 with the computational device 501 shown and described under FIG. 5.

In further embodiments, the eyewear device maybe equipped with one or more USB ports for transporting stored data and downloading software programs and apps.

In embodiments, the system depicted in FIG. 6 includes one or more eyewear devices having integrated, customized, controllable and cross-linked Mobile/Tablet device applications for training and analytics.

The foregoing description of the eyewear device and system for sensory training has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description may describe the various embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. The described operations may be embodied in software, firmware, hardware, or any combinations thereof.

The software components or functions described in this application may be implemented as software code to be executed by one or more processors using any suitable computer language. The software code may be stored as a series of computer-readable instructions on a non-transitory computer-readable medium, such as a random access memory (RAM), a read-only memory (ROM), a magnetic medium such as a hard-drive or a floppy disk, or an optical medium such as a CD-ROM. Any such computer-readable medium may also reside on or within a single computational apparatus, and may be present on or within different computational apparatuses within a system or network.

Any of the steps, operations, or processes described herein may be performed or implemented with one or more hardware or software modules, alone or in combination with other devices. In one embodiment, a software module is implemented with a computer program product comprising a non-transient computer-readable medium containing computer program code, which can be executed by a computer processor for performing any or all of the steps, operations, or processes described.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the subject matter, which is set forth in the following claims.

Throughout the specification and claims unless the contents requires otherwise the word ‘comprise’ or variations such as ‘comprises’ or ‘comprising’ will be understood to imply the inclusion of a stated integer or group of integers but not the exclusion of any other integer or group of integers.

I claim:

1. An eyewear device for mind body sensory integration training comprising:
at least one lens fabricated from polymer-dispersed liquid crystals (PDLC) on at least one substrate, the substrate comprising a compliant polymer; wherein:
(i) Responsive to being in an ‘OFF’ state, said at least one lens is opaque; and
(ii) Responsive to being in an ‘ON’ state, said at least one lens is at least partially transparent;
Said eyewear device further comprising:
(iii) A frame configured for wear by the user to which said at least one lens is fixedly attached;
(iv) A plurality of integrated touch or touch-sensitive controls for selecting from a plurality of programs for control of said at least one lens; and
(v) An integrated Wi-Fi transceiver, for receiving program instructions transmitted by a control device wirelessly coupled to said eyewear device.

2. The eyewear device of claim 1, where said at least one lens is configured to cycle between the ‘OFF’ state and the ‘ON’ state.

3. The device of claim 1, further comprising:
(i) At least one integrated display; and
(ii) At least one LED for indicating device state.

4. The eyewear device of claim 1, further comprising a power supply and a power system for delivering power from said supply to said eyewear device.

5. The eyewear device of claim 4, further comprising a charge controller and protection to protect said power supply and to charge said power supply via a charging port.

6. The eyewear device of claim 5, further includes a control device comprising of:
(i) A local memory storing the control programs;
(ii) A local processor for executing the control programs.

7. The eyewear device of claim 6, wherein the control device wirelessly coupled to said eyewear device comprises at least one of a wireless communications device and a computational device.

8. The eyewear device of claim 6, wherein the wireless communication is established through WIFI, Bluetooth and infrared.

9. The eyewear device of claim 7, wherein at least one of said wireless communications device and said computational device comprise:
(i) A memory for storing the control program; and
(ii) A processor for executing the control programs.

10. The eyewear device of claim 6, wherein the control device wirelessly coupled to said eyewear device is operative to simultaneously control a plurality of eyewear devices, each eyewear device being simultaneously coupled to the control device.

11. The eyewear device of claim 1, further comprising shielding to render said eyewear device water-resistant, thereby shielding at least said lens from moisture damage.

12. The eyewear device of claim 1, further comprising Program instructions embodied on a non-transitory medium for control said at least one lens.

13. The eyewear device of claim 1, wherein said at least one lens is provided in a plurality of colors.

14. The eyewear device of claim 1, wherein said at least one HRV (Heart Rate Variability) sensor imbedded into the frame.

15. The eyewear device of claim 1, wherein said at least one micro speaker imbedded into the frame.
